(19) United States
(12) Patent Application Publication
(10) Pub. No.: US 2017/0219597 A1
(43) Pub. Date: Aug. 3, 2017

(54) METHODS AND COMPOSITIONS FOR SELECTIVE DETECTION OF HOMOSYSTEINE

(71) Applicant: Georgia State University Research Foundation, Inc., Atlanta, GA (US)

(72) Inventors: Binghe Wang, Marietta, GA (US); Hanjing Peng, Lutherville, MD (US); Ke Wang, Chamblee, GA (US)

(21) Appl. No.: 15/320,392

(22) PCT Filed: Jun. 25, 2015
(86) PCT No.: PCT/US15/37784

§ 371 Date: Dec. 20, 2016

Related U.S. Application Data
(60) Provisional application No. 62/017,414, filed on Jun. 26, 2014.

(51) Int. Cl. G01N 33/68

(52) U.S. Cl. CPC G01N 33/6815; G01N 2800/2821; G01N 2800/50

(57) ABSTRACT

Compositions and methods have been developed for the detection of homocysteine. For example, a fluorescent probe can selectively detect homocysteine based on the redox reaction between the azido group and homocysteine. The fluorescent response is selective for homocysteine over other biologically abundant thiols such as cysteine and glutathione. In addition, a linear calibration curve can be obtained for quantitative analysis in phosphate buffer and plasma.

### FIELD OF THE INVENTION

The disclosed invention is generally in the field of metabolite detection and specifically in the area of detection of homocysteine.

### BACKGROUND OF THE INVENTION

Plasma homocysteine is an important risk factor for various diseases, such as cardiovascular disease and Alzheimer's disease. Therefore, methods for its detection and concentration determination are useful in research and clinical settings. Biological thiols such as cysteine (Cys), homocysteine (Hcy) and glutathione (GSH) play versatile roles in various processes, including protein structure and functions, antioxidant activity, and redox signaling. Hcy is a key intermediate generated during the biosynthesis of cysteine (Chen et al., Faseb J, 2010, 24, 2804-2817; Selhub, Annu Rev Nutr, 1999, 19, 217-246). Plasma hey exists in three major forms including free reduced (˜1%), free oxidized (30% low molecular disulfides) and protein-bound oxidized (70%) forms, with the two oxidized forms being predominant (Ueland, Clin Chem, 1995, 41, 340-342; Chambers et al., Circ Res, 2001, 89, 187-192). The sum of all forms, total Hcy (tHcy) has been identified as an independent risk factor for cardiovascular disease (CVD) (Refsum and Smith, New Engl J Med, 2006, 355, 207-207; Refsum et al., Annu Rev Med, 1998, 49, 31-62; Boushey et al., Jama-J Am Med Assoc, 1995, 274, 1049-1057), diabetes (Wijekoon et al., 2007, 35, 1175-1179), renal disease (van Guldener, Nephrol. Dial. Transpl., 2006, 21, 1161-1166; Chauveau et al., Miner. Electrol. Metab., 1996, 22, 106-109), and neurodegenerative diseases such as Alzheimer's disease (AD) (Miller et al., Faseb J, 1992, 6, A1215-A1215; Malinow et al., Circulation, 1999, 99, 178-182). Impaired methionine and Hcy metabolism leads to hyperhomocysteiemia (HHcy), which is demonstrated by an elevated level of plasma tHcy (Miller et al., Faseb J, 1992, 6, A1215-A1215). According to the American Heart Association (AHA) advisory statement, normal tHcy concentrations range from 5-15 μM; moderate, intermediate, and severe hyperhomocysteinemia refer to concentrations between 16 and 30, between 31 and 100, and >100 μM, respectively, and are essentially pathognomonic (Malinow et al., Circulation, 1999, 99, 178-182). As a result, accurate determinations of Hcy in biological systems such as blood plasma and urine are of great importance in diagnostic applications.

Due to high degree of similarity in both structure and chemical properties between Hcy and other thiols such as cysteine, the selective detection of Hcy has never been a trivial issue. Current detection methods for Hcy analysis involve the derivatization at the sulfhydryl group using electrophilic fluorogenic reagents (Ueland et al., Clin Chem, 1993, 39, 1764-1779; Kusmierek et al., Anal Bioanal Chem, 2006, 385, 855-860; Chen et al., Chem Soc Rev, 2010, 39, 2120-2135). However, due to the lack of selectivity among different biological thiols, these methods require tedious separating techniques such as GC and HPLC. This has led to an increased cost and reduced efficiency and thus limited the applicability of Hcy as an important biomarker. Therefore, selective recognition of homocysteine over other biological thiols is of great interest. Selective fluorescent probes based on cyclization mechanisms using both the amino and sulfhydryl groups have been reported (Chen et al., Inorg. Chem., 2007, 46, 11075-11081; Yang et al., Angew. Chem. Int. Ed., 2011, 50, 10690-10693; Guo et al., Chem. Sci., 2012, 3, 2760-2765). In addition, the Strongin group has found a method for selective Hcy detection using a commercially available reagent based on a radical mechanism (Strongin et al., J Am Chem Soc, 2004, 126, 3400-3401; Rusin et al., J Am Chem Soc, 2005, 127, 15949-15958; Escobedo et al., Nat Protoc, 2006, 1, 2759-2762). However, these methods are either selective for cysteine or require drastic conditions such as boiling. This has also limited their applications.

It is therefore an object of the present invention to provide methods and compositions for selective detection of homocysteine.

It is also an object of the present invention to provide methods and compositions for efficient detection of homocysteine.

It is also an object of the present invention to provide methods and compositions for rapid detection of homocysteine.

It is also an object of the present invention to provide methods and compositions for easy detection of homocysteine.

It is also an object of the present invention to provide methods and compositions for selective, efficient, rapid, easy, or a combination thereof, detection of homocysteine.

### BRIEF SUMMARY OF THE INVENTION

Compositions and methods have been developed for the detection of homocysteine. For example, a fluorescent probe can selectively detect homocysteine based on the redox reaction between the azido group and homocysteine. The fluorescent response is selective for homocysteine over other biologically abundant thiols such as cysteine and glutathione. In addition, a linear calibration curve can be obtained for quantitative analysis in phosphate buffer and plasma.

Disclosed are methods and compositions for measuring the amount of homocysteine in samples, detecting or diagnosing high levels of homocysteine in subjects, monitoring homocysteine levels in samples and subjects, identifying subjects as at risk of disease based on detection of high levels of homocysteine in the subject, and treating subjects for a disease or to reduce the risk of the disease based on detection of high levels of homocysteine in the subject. The methods and compositions involve use of a sulfonyl azide compound of formula I

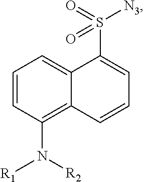

where R₁ is —CH₃, —CH₂—CH₃, or —CH₂—CH₂—CH₃, and R₂ is —CH₂—CH₂—N(CH₃)₂, —CH₂—N(CH₃)₂, —CH₂—CH₂—CH₂—N(CH₃)₂, —CH₃, —CH₂—CH₃, or —CH₂—CH₂—CH₃.

These compounds undergo redox reactions with homocysteine with the result that the weak fluorescence of the compound is significantly increased after the reaction. The increased fluorescence indicates the presence of homocysteine and can be used to quantitate the level of homocysteine. Some of these compounds, such as the compound where R₁ is —CH₃ and R₂ is —CH₂—CH₂—N(CH₃)₂, and where R₁ and R₁ are —CH₃, selectively react with homocysteine as compared to other common biological thiols such as cysteine and glutathione. This selectivity allows more specific detection of homocysteine in some types of samples, such as biological samples, and increases the accuracy of quantitation of homocysteine levels.

In some forms, the method involves incubating a sample and a sulfonyl azide compound and measuring fluorescence of a reaction product of the compound and homocysteine. The measured fluorescence level indicates the amount of homocysteine in the sample.

In some forms, the method involves incubating a sample and a sulfonyl azide compound, measuring fluorescence of a reaction product of the compound and homocysteine, and comparing the measured fluorescence level to calibration curve of the fluorescence level of different concentrations of homocysteine. The comparison indicates the amount of homocysteine in the sample.

In some forms, the method involves incubating a sample from a subject and a sulfonyl azide compound, measuring fluorescence of a reaction product of the compound and homocysteine, and identifying the subject as at risk of disease based on the indicated amount of homocysteine in the sample.

In some forms, the method involves incubating a sample from a subject and a sulfonyl azide compound, measuring fluorescence of a reaction product of the compound and homocysteine, identifying the subject as at risk of disease based on the indicated amount of homocysteine in the sample, and treating the subject for the disease or to reduce the risk of the disease if the subject is identified as at risk of disease based on the indicated amount of homocysteine in the sample.

In some forms, R₁ is —CH₃ and R₂ is —CH₂—CH₂—N(CH₃)₂ in the sulfonyl azide compound. In some forms, R₁ is —CH₃ and R₂ is —CH₃ in the sulfonyl azide compound.

In some forms, the sample is a body fluid sample of a subject. In some forms, the body sample fluid is serum. In some forms, the serum is exposed to reducing conditions prior to the incubation. In some forms, the exposure to reducing conditions is accomplished by exposing the serum to gel containing tris(2-carboxyethyl)phosphine) (TCEP). In some forms, the serum is diluted prior to the incubation.

In some forms, the incubation is in the presence of a transition metal salt. In some forms, the incubation is in the presence of a Zinc salt, a Cadmium salt, a Copper salt, a Lead salt, a Mercury salt, a Silver salt, or a combination thereof. In some forms, the incubation is in the presence of a Zinc(II) salt, a Cadmium(II) salt, a Copper(II) salt, a Lead(II) salt, a Mercury(II) salt, a Silver(I) salt, or a combination thereof. In some forms, the Zinc salt is ZnCl₂.

In some forms, the incubation and measurement is carried out at a pH of 6 to 9. In some forms, the incubation and measurement is carried out at a pH of 7 to 8. In some forms, the incubation and measurement is carried out at a pH of 7.4. In some forms, the measurement is carried out at a pH of 7.4.

In some forms, the measured fluorescence level indicates the amount of homocysteine in the sample by comparing the measured fluorescence level to calibration curve of the fluorescence level of different concentrations of homocysteine.

In some forms, the method includes identifying the subject as at risk of disease based on the indicated amount of homocysteine in the sample. In some forms, the disease is cardiovascular disease or Alzheimer's disease.

In some forms, the method includes treating the subject for the disease or to reduce the risk of the disease if the subject is identified as at risk of disease based on the indicated amount of homocysteine in the sample.

In some forms, the subject is identified as at risk of disease if the indicated amount of homocysteine in the sample is greater than 14 moles/liter. In some forms, the subject is identified as at intermediate risk of disease if the indicated amount of homocysteine in the sample is greater than 29 μmoles/liter.

Also disclosed are kits for measuring the amount of homocysteine in a sample. The kit can include a sulfonyl azide compound of formula I

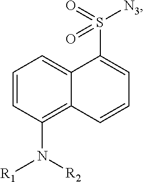

where R₁ is —CH₃, —CH₂—CH₃, or —CH₂—CH₂—CH₃, and R₂ is —CH₂—CH₂—N(CH₃)₂, —CH₂—N(CH₃)₂, —CH₂—CH₂—CH₂—N(CH₃)₂, —CH₃, —CH₂—CH₃, or —CH₂—CH₂—CH₃, and a transition metal salt, such as a Zinc salt, a Cadmium salt, a Copper salt, a Lead salt, a Mercury salt, a Silver salt, or a combination thereof.

Also disclosed is the compound of formula II:

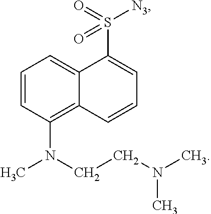 [SMILES: CN(C)CCN(C)c1cccc2c(S(=O)(=O)N=[N+]=[N-])cccc12]

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A is a graph of fluorescence spectrum of DN-2 in the absence and presence of different amino thiols. FIG. 1B is a graph showing time dependent fluorescence emission (517 nm) of DN-2 in the presence of different Hcy and Cys, and comparison of fluorescence intensity change with the addition of different amino thiols. DN-2 120 μM, amino thiols 100 μM in 100 mM sodium phosphate buffer at pH 7.4 with 10% ethanol, fluorescence spectrum and pictures were recorded 1 h after the addition of thiols.

FIG. 2A is a graph showing the quantum yield determination of DN-2 (1) and DA-2 (2) in acetonitrile. FIG. 2B is a graph showing the quantum yield determination of DN-2 (1) and DA-2 (2) in water.

FIG. 3A is a graph of fluorescence response of DN-2 to amino acids. FIG. 3B is a graph of fluorescence response of DN-2 to Hcy in the presence of amino acids. DN-2 120 μM, amino thiols 100 μM in 100 mM sodium phosphate buffer at pH 7.4 with 10% ethanol, fluorescence intensities were recorded 1 h after the addition of amino acids. Data represents the average of three independent experiments.

FIG. 4 is a calibration curve for homocysteine, DN-2 120 μM, Hcy 100 μM in 100 mM sodium phosphate buffer at pH 7.4, with 10% EtOH; fluorescence intensities at 517 nm were recorded 60 min after the addition of Hcy. Data represents the average of three independent experiments.

FIG. 5 is a graph showing a calibration curve of homocysteine in the presence of 200 and 250 μM of cysteine. DN-2 120 μM, Hcy 0-50 μM in 100 mM sodium phosphate buffer at pH 7.4, with 10% EtOH; fluorescence intensities at 517 nm were recorded 60 min after the addition of Hcy. Data represents the average of three independent experiments.

FIG. 6 is a graph of fluorescence response of DN-2 to Hey in the presence of different concentrations of Zn²⁺. DN-2 120 μM, Hcy 100 μM in 100 mM phosphate buffer with 10% EtOH at pH 7.4. Fluorescence was recorded on a fluorometer after reaction for 120 min in the presence of 0, 50, 100, 200 and 300 μM of ZnCl₂, respectively. Data represents the average of three independent experiments.

FIG. 7A is a calibration curve for Hcy. DN-2, 120 μM, Hcy, 0-100 μM in diluted deproteinized FBS, fluorescence intensities at 517 nm were recorded 180 min after addition of Hcy. Data represents the average of three independent experiments. FIG. 7B is a graph showing time-dependent fluorescence response of DN-2 (120 μM) to Hcy (100 μM) or Cys (100 μM) in diluted deproteinized FBS.

FIG. 8A is a graph of a titration curve of 5 mM cysteine hydrochloride. FIG. 8B is a graph showing calculation of the sulfhydryl group pKa of cysteine. FIG. 8C is a graph of a titration curve of 5 mM homocysteine hydrochloride. FIG. 8D is a graph showing calculation of the sulfhydryl group pKa of homocysteine.

FIG. 9 is a diagram of the proposed mechanism of the reaction of DN-2 with homocysteine.

FIG. 10 is a graph showing time dependent fluorescence emission (517 nm) of 1,5-DNS-Az in the presence of Hcy and Cys. DNS-Az 120 μM, amino thiols 100 μM in 100 mM sodium phosphate buffer at pH 7.4 with 10% EtOH.

FIG. 11 is a graph showing pH dependent fluorescence response of DN-2 to thiols. Fluorescence intensity was read on a microplate reader. Excitation filter 340 nm, Emission filter 535 nm. DN-2 100 μM, thiols 100 μM in 100 mM sodium phosphate buffer at pH values of 5.3, 6.2, 7.0, 7.5, 8.3, 9.0, 10.5, and 11.5.

FIG. 12 is a graph showing reaction time profile at 37° C. DN-2 120 μM, amino thiols 100 μM in 100 mM sodium phosphate buffer at pH 7.5, 37° C., fluorescence intensities at 517 nm were recorded at each time point.

### DETAILED DESCRIPTION OF THE INVENTION

The disclosed method and compositions may be understood more readily by reference to the following detailed description of particular embodiments and the Example included therein and to the Figures and their previous and following description.

Compositions and methods have been developed for the detection of homocysteine. A fluorescent probe selectively detects homocysteine based on the redox reaction between the azido group and homocysteine. The fluorescence response is selective for homocysteine over other biologically abundant thiols such as cysteine and glutathione. The fluorescence response is selective for homocysteine over other biologically abundant thiols such as cysteine and glutathione. In addition, a linear calibration curve can be obtained for quantitative analysis in phosphate buffer and plasma.

Homocysteine is a very important biomarker for the diagnosis and prognosis of various diseases such as cardiovascular diseases and neurodegenerative diseases. However, due to the structural similarity of homocysteine and cysteine, direct detection of this biomarker still remains a major challenge. A new redox sensitive fluorescent probe was developed for quantitative homocysteine analysis. The sensing reaction is selective for homocysteine over other biological thiols such as cysteine and GSH. In addition, a linear calibration curve could be obtained in both buffer and diluted deproteinized FBS with a detection limits at 10 μM. Endogenous cysteine level fluctuation (200-250 μM) can also be tolerated to provide a good estimation of homocysteine concentration. This probe will be very useful for the selective detection of Hey in complex biological samples.

Disclosed are methods and compositions for measuring the amount of homocysteine in samples, detecting or diagnosing high levels of homocysteine in subjects, monitoring homocysteine levels in samples and subjects, identifying subjects as at risk of disease based on detection of high levels of homocysteine in the subject, and treating subjects for a disease or to reduce the risk of the disease based on detection of high levels of homocysteine in the subject. The methods and compositions involve use of a sulfonyl azide compound of formula I

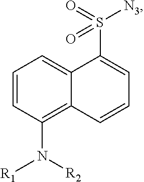

where R₁ is —CH₃, —CH₂—CH₃, or —CH₂—CH₂—CH₃, and R₂ is —CH₂—CH₂—N(CH₃)₂, —CH₂—N(CH₃)₂, —CH₂—CH₂—CH₂—N(CH₃)₂, —CH₃, —CH₂—CH₃, or —CH₂—CH₂—CH₃.

These compounds undergo redox reactions with homocysteine with the result that the weak fluorescence of the compound is significantly increased after the reaction. The increased fluorescence indicates the presence of homocysteine and can be used to quantitate the level of homocysteine. Some of these compounds, such as the compound where R₁ is —CH₃ and R₂ is —CH₂—CH₂—N(CH₃)₂, and where R₁ and R₁ are —CH₃, selectively react with homocysteine as compared to other common biological thiols such as cysteine and glutathione. This selectivity allows more specific detection of homocysteine in some types of samples, such as biological samples, and increases the accuracy of quantitation of homocysteine levels.

As used herein, “selective” refers to a reaction, interaction, or signal that occurs more or at a higher level than one or more other reactions, interactions, or signals. For example, a fluorescent probe selectively detects homocysteine compared to cysteine and glutathione by reacting more and/or producing greater fluorescence with homocysteine than with cysteine and glutathione. The selectivity can be characterized by the relative difference in reaction, interaction, or signal. For example, a selective reaction, interaction, or signal produces 1.5, 2, 2.5, 3, 3.5, 4, 4.5, 5, 5.5, 6, 6.5, 7, 7.5, 8, 8.5, 9, 9.5, 10, 12, 14, 16, 18, 20, 25, 30, 35, 40, 45, 50, 60, 70, 80, 90, 100, or more times the reaction, interaction, or signal compared with one or more other reactions, interactions, or signals.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a sulfonyl azide compound is disclosed and discussed and a number of modifications that can be made to a number of molecules including the sulfonyl azide compound are discussed, each and every combination and permutation of sulfonyl azide compound and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Compounds

In one aspect described herein are compounds having the formula I.

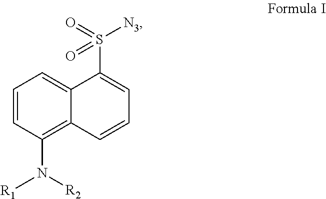

In some forms of Formula I, R₁ is —CH₃, —CH₂—CH₃, or —CH₂—CH₂—CH₃, and R₂ is —CH₂—CH₂—N(CH₃)₂, —CH₂—N(CH₃)₂, —CH₂—CH₂—CH₂—N(CH₃)₂, —CH₃, —CH₂—CH₃, or —CH₂—CH₂—CH₃. Preferred are compounds where R₁ is —CH₃ and R₂ is —CH₂—CH₂—N(CH₃)₂ or where R₁ is —CH₃ and R₂ is —CH₃.

These compounds undergo redox reactions with homocysteine with the result that the weak fluorescence of the compound is significantly increased after the reaction. The increased fluorescence indicates the presence of homocysteine and can be used to quantitate the level of homocysteine. Some of these compounds, such as the compound where R₁ is —CH₃ and R₂ is —CH₂—CH₂—N(CH₃)₂, and where R₁ and R₁ are —CH₃, selectively react with homocysteine as compared to other common biological thiols such as cysteine and glutathione. This selectivity allows more specific detection of homocysteine in some types of samples, such as biological samples, and increases the accuracy of quantitation of homocysteine levels.

Preferred is the compound of formula II:

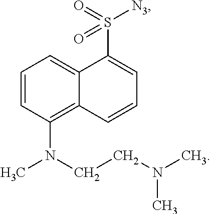 [SMILES: CN(C)CCN(C)c1cccc2c(S(=O)(=O)N=[N+]=[N-])cccc12]

### B. Samples

Any sample can be used with the disclosed methods. Examples of suitable samples include bodily fluids, biopsy samples, whole cell samples, tissue samples, culture samples, environmental samples, or a combination. Numerous other sources of nucleic acid samples are known or can be developed and any can be used with the disclosed method. Generally, it is useful to use a genomic sample from cells, tissues, subjects, that are relevant to the status being assessed.

The source, identity, and preparation of many such samples are known. The sample can be, for example, a sample from one or more cells, tissue, skin, lung, head, neck, prostate, breast, ovary, brain, liver, stomach, intestine, kidney, testicle, cervix, uterus, spleen, bone, throat, esophagus, muscle, or bodily fluids such as blood, urine, semen, lymphatic fluid, cerebrospinal fluid, or amniotic fluid, or other biological samples, such as tissue culture cells, buccal swabs, mouthwash, stool, tissues slices, and biopsy aspiration. Types of useful samples include blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, crude cell lysate samples, forensic samples, infection samples, and/or nosocomial infection samples.

### C. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for measuring the amount of homocysteine in a sample. The kit can include a sulfonyl azide compound of formula I

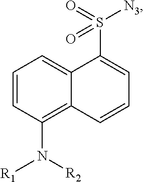

where R₁ is —CH₃, —CH₂—CH₃, or —CH₂—CH₂—CH₃, and R₂ is —CH₂—CH₂—N(CH₃)₂, —CH₂—N(CH₃)₂, —CH₂—CH₂—CH₂—N(CH₃)₂, —CH₃, —CH₂—CH₃, or —CH₂—CH₂—CH₃, and a transition metal salt, such as a Zinc salt, a Cadmium salt, a Copper salt, a Lead salt, a Mercury salt, a Silver salt, or a combination thereof. In some forms, the incubation is in the presence of a Zinc(II) salt, a Cadmium(II) salt, a Copper(II) salt, a Lead(II) salt, a Mercury(II) salt, a Silver(I) salt, or a combination thereof. A preferred Zinc salt is ZnCl₂. The kits also can contain a reducing reagent. For example, the kit can include a gel containing tris(2-carboxyethyl)phosphine) (TCEP), such as Immobilized TCEP Disulfide Reducing Gel (Pierce, 77712).

### D. Mixtures

Disclosed are mixtures formed by performing or preparing to perform the disclosed method. For example, disclosed are mixtures comprising a sample and a sulfonyl azide compound of formula I

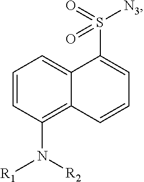

where R₁ is —CH₃, —CH₂—CH₃, or —CH₂—CH₂—CH₃, and R₂ is —CH₂—CH₂—N(CH₃)₂, —CH₂—N(CH₃)₂, —CH₂—CH₂—CH₂—N(CH₃)₂, —CH₃, —CH₂—CH₃, or —CH₂—CH₂—CH₃. As another example, disclosed are mixtures comprising a sample, a sulfonyl azide compound, and a transition metal salt, such as a Zinc salt, a Cadmium salt, a Copper salt, a Lead salt, a Mercury salt, a Silver salt, or a combination thereof. In some forms, the transition metal salt can be a Zinc(II) salt, a Cadmium(II) salt, a Copper(II) salt, a Lead(II) salt, a Mercury(II) salt, a Silver(I) salt, or a combination thereof.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### E. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Methods

Disclosed are methods for measuring the amount of homocysteine in samples, detecting or diagnosing high levels of homocysteine in subjects, monitoring homocysteine levels in samples and subjects, identifying subjects as at risk of disease based on detection of high levels of homocysteine in the subject, and treating subjects for a disease or to reduce the risk of the disease based on detection of high levels of homocysteine in the subject. The methods involve use of a sulfonyl azide compound of formula I

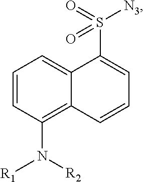

where R₁ is —CH₃, —CH₂—CH₃, or —CH₂—CH₂—CH₃, and R₂ is —CH₂—CH₂—N(CH₃)₂, —CH₂—N(CH₃)₂, —CH₂—CH₂—CH₂—N(CH₃)₂, —CH₃, —CH₂—CH₃, or —CH₂—CH₂—CH₃.

These compounds undergo redox reactions with homocysteine with the result that the weak fluorescence of the compound is significantly increased after the reaction. The increased fluorescence indicates the presence of homocysteine and can be used to quantitate the level of homocysteine. Some of these compounds, such as the compound where R₁ is —CH₃ and R₂ is —CH₂—CH₂—N(CH₃)₂ (Formula II), and where R₁ and R₁ are —CH₃, selectively react with homocysteine as compared to other common biological thiols such as cysteine and glutathione. This selectivity allows more specific detection of homocysteine in some types of samples, such as biological samples, and increases the accuracy of quantitation of homocysteine levels.

In some forms, the method involves incubating a sample and a sulfonyl azide compound and measuring fluorescence of a reaction product of the compound and homocysteine. The measured fluorescence level indicates the amount of homocysteine in the sample.

The incubation can be carried out under conditions suitable for reacting the sulfonyl azide compound with homocysteine. Preferred incubations are those that increase or maximize the selectivity of the reaction to homocysteine. Examples of such incubation conditions (and how to determine such incubation conditions) are described in the examples. For example, the incubation can be carried out at a pH of 6 to 9. In some forms, the incubation can be carried out at a pH of 7 to 8. In some forms, the incubation can be carried out at a pH of 7.2 to 7.6. In some forms, the incubation can be carried out at a pH of 7.3 to 7.5. In some forms, the incubation can be carried out at a pH of 7.4.

In some forms, the incubation can be carried out at a temperature of from 15° C. to 40° C. In some forms, the incubation can be carried out at a temperature of from 22° C. to 37° C. In some forms, the incubation can be carried out at a temperature of from 25° C. to 37° C. In some forms, the incubation can be carried out at a temperature of from 30° C. to 37° C. In some forms, the incubation can be carried out at a temperature of 37° C.

In some forms, the incubation can be carried out for 15 to 180 minutes. In some forms, the incubation can be carried out for 20 to 120 minutes. In some forms, the incubation can be carried out for 30 to 60 minutes. In some forms, the incubation can be carried out for 30 to 45 minutes. In some forms, the incubation can be carried out for 30 minutes.

In some forms, the incubation is in the presence of a transition metal salt. In some forms, the incubation is in the presence of a Zinc salt, a Cadmium salt, a Copper salt, a Lead salt, a Mercury salt, a Silver salt, or a combination thereof. In some forms, the incubation is in the presence of a Zinc(II) salt, a Cadmium(II) salt, a Copper(II) salt, a Lead(II) salt, a Mercury(II) salt, a Silver(I) salt, or a combination thereof. In some forms, the Zinc salt is ZnCl₂. Sulfides of some transition metals are especially insoluble and so are preferred. For example, ZnS (Kₛₚ=2×10⁻²⁵), CdS (Kₛₚ=8×10⁻²⁸), CuS (Kₛₚ=6×10⁻³⁷), PbS (Kₛₚ=3×10⁻²), HgS (Kₛₚ=2×10⁻⁵³), and Ag₂S (Kₛₚ=6×10⁻⁵¹).

In some forms, the incubation can be carried out at a transition metal concentration of from 20 μM to 400 μM. In some forms, the incubation can be carried out at a transition metal concentration of from 50 μM to 300 μM. In some forms, the incubation can be carried out at a transition metal concentration of from 100 μM to 300 μM. In some forms, the incubation can be carried out at a transition metal concentration of from 150 μM to 300 μM. In some forms, the incubation can be carried out at a transition metal concentration of from 200 μM to 300 μM. In some forms, the incubation can be carried out at a transition metal concentration of from 250 μM to 300 μM. In some forms, the incubation can be carried out at a transition metal concentration of 250 μM.

In some forms, the incubation can be carried out at a Zinc concentration of from 20 μM to 400 μM. In some forms, the incubation can be carried out at a Zinc concentration of from 50 μM to 300 μM. In some forms, the incubation can be carried out at a Zinc concentration of from 100 μM to 300 μM. In some forms, the incubation can be carried out at a Zinc concentration of from 150 μM to 300 μM. In some forms, the incubation can be carried out at a Zinc concentration of from 200 μM to 300 μM. In some forms, the incubation can be carried out at a Zinc concentration of from 250 μM to 300 μM. In some forms, the incubation can be carried out at a Zinc concentration of 250 μM.

The measurement can be made continuously, episodically, at particular time points, at an initial (or starting) and a final time point, at a final time point, or a combination. The measurement (as a whole or any specific time point measurement(s)) can be made during, overlapping with, or following the incubation (or a combination). Preferably the measurement is made at a single final time point following (or at the end of) the incubation. The measurement can be made under the same or different conditions form the incubation.

In some forms, the measurement can be carried out at a pH of 6 to 9. In some forms, the measurement can be carried out at a pH of 7 to 8. In some forms, the measurement can be carried out at a pH of 7.2 to 7.6. In some forms, the measurement can be carried out at a pH of 7.3 to 7.5. In some forms, the measurement can be carried out at a pH of 7.4.

In some forms, the measurement can be carried out at a temperature of from 15° C. to 40° C. In some forms, the measurement can be carried out at a temperature of from 22° C. to 37° C. In some forms, the measurement can be carried out at a temperature of from 25° C. to 37° C. In some forms, the measurement can be carried out at a temperature of from 30° C. to 37° C. In some forms, the measurement can be carried out at a temperature of 37° C.

In some forms, the measurement can be carried out for 15 to 180 minutes. In some forms, the measurement can be carried out for 20 to 120 minutes. In some forms, the measurement can be carried out for 30 to 60 minutes. In some forms, the measurement can be carried out for 30 to 45 minutes. In some forms, the measurement can be carried out for 30 minutes.

In some forms, the measurement can be carried out at a transition metal concentration of from 20 μM to 400 μM. In some forms, the measurement can be carried out at a transition metal concentration of from 50 μM to 300 μM. In some forms, the measurement can be carried out at a transition metal concentration of from 100 μM to 300 μM. In some forms, the measurement can be carried out at a transition metal concentration of from 150 μM to 300 μM. In some forms, the measurement can be carried out at a transition metal concentration of from 200 μM to 300 μM. In some forms, the measurement can be carried out at a transition metal concentration of from 250 μM to 300 μM. In some forms, the measurement can be carried out at a transition metal concentration of 250 μM.

In some forms, the measurement can be carried out at a Zinc concentration of from 20 μM to 400 μM. In some forms, the measurement can be carried out at a Zinc concentration of from 50 μM to 300 μM. In some forms, the measurement can be carried out at a Zinc concentration of from 100 μM to 300 μM. In some forms, the measurement can be carried out at a Zinc concentration of from 150 μM to 300 μM. In some forms, the measurement can be carried out at a Zinc concentration of from 200 μM to 300 μM. In some forms, the measurement can be carried out at a Zinc concentration of from 250 μM to 300 μM. In some forms, the measurement can be carried out at a Zinc concentration of 250 μM.

In some forms, the measurement can be made at a wavelength of from 460 nm to 570 nm. In some forms, the incubation can be measurement can be made at a wavelength of from 470 nm to 560 nm. In some forms, the incubation can be measurement can be made at a wavelength of from 480 nm to 550 nm. In some forms, the incubation can be measurement can be made at a wavelength of from 490 nm to 540 nm. In some forms, the incubation can be measurement can be made at a wavelength of from 500 nm to 530 nm. In some forms, the incubation can be measurement can be made at a wavelength of from 510 nm to 520 nm. In some forms, the incubation can be measurement can be made at a wavelength of 517 nm.

The sample can be treated in any useful manner prior to the incubation. For example, it may be useful to partially purify, clarify, deproteinize, or dilute the sample prior to incubation. In some forms, the sample is exposed to reducing conditions prior to the incubation. In some forms, the sample is diluted prior to the incubation. In some forms, the sample is deproteinized prior to the incubation. In some forms, the sample is clarified prior to the incubation. Generally, clarification involves removing solid components of a sample by centrifugation. In some forms, the body fluid sample is exposed to reducing conditions prior to the incubation. In some forms, the body fluid sample is diluted prior to the incubation. In some forms, the body fluid sample is deproteinized prior to the incubation. In some forms, the body fluid sample is clarified prior to the incubation. In some forms, the blood sample is exposed to reducing conditions prior to the incubation. In some forms, the blood sample is diluted prior to the incubation. In some forms, the blood sample is deproteinized prior to the incubation. In some forms, the blood sample is clarified prior to the incubation. In some forms, the serum is exposed to reducing conditions prior to the incubation. In some forms, the serum sample is diluted prior to the incubation. In some forms, the serum sample is deproteinized prior to the incubation. In some forms, the serum sample is clarified prior to the incubation.

In some samples, homocysteine may be oxidized, such as Hcy-S—S-Hcy, Hcy-S—S-Cys, or Hcy-S—S-peptide. To improve the method, the sample can be pre-reduced prior to the incubation. For example, the sample can be exposed to TCEP gel (a kind of beads with TCEP (tris(2-carboxyethyl)phosphine) immobilized onto it) to release free sulfhydryl group. A useful TCEP gel is available from Pierce, catalogue number 77712.

The disclosed methods can include other steps and purposes beyond the detection and measurement of homocysteine. For example, in some forms, the method can involve incubating a sample and a sulfonyl azide compound, measuring fluorescence of a reaction product of the compound and homocysteine, and comparing the measured fluorescence level to calibration curve of the fluorescence level of different concentrations of homocysteine. The comparison indicates the amount of homocysteine in the sample. The calibration curve can be preexisting or created at or near the same time as the assay. It is preferred that the calibration curve be generated under the same or similar conditions of incubation and measurement as those used in the assay.

In some forms, the method can involve incubating a sample from a subject and a sulfonyl azide compound, measuring fluorescence of a reaction product of the compound and homocysteine, and identifying the subject as at risk of disease based on the indicated amount of homocysteine in the sample. Some risk of disease is present if the indicated amount of homocysteine in the sample is greater than 14 moles/liter but less than 29 moles/liter. An intermediate risk of disease risk of disease is present if the indicated amount of homocysteine in the sample is greater than 29 moles/liter but less than 100 moles/liter. A high risk of disease risk of disease is present if the indicated amount of homocysteine in the sample is greater than 100 μmoles/liter. Diseases associated with high levels of homocysteine include cardiovascular disease or Alzheimer's disease.

In some forms, the method can involve incubating a sample from a subject and a sulfonyl azide compound, measuring fluorescence of a reaction product of the compound and homocysteine, identifying the subject as at risk of disease based on the indicated amount of homocysteine in the sample, and treating the subject for the disease or to reduce the risk of the disease if the subject is identified as at risk of disease based on the indicated amount of homocysteine in the sample.

A high level of homocysteine makes a person more prone to endothelial injury, which leads to vascular inflammation, which in turn may lead to atherogenesis, which can result in ischemic injury. Hyperhomocysteinemia is therefore a possible risk factor for coronary artery disease. Coronary artery disease occurs when an atherosclerosis leads to occlusion of the lumina of the coronary arteries. These arteries supply the heart with oxygenated blood.

Hyperhomocysteinemia has been correlated with the occurrence of blood clots, heart attacks and strokes, though it is unclear whether hyperhomocysteinemia is an independent risk factor for these conditions. It can cause miscarriage and/or pre-eclampsia in pregnant women, and can lead to birth defects.

Hyperhomocysteinemia or hyperhomocysteinaemia is a medical condition characterized by an abnormally high level of homocysteine in the blood, conventionally described as above 15 μmol/L (Guo et al., The Indian journal of medical research 129 (3): 279-84 (2009)). As a consequence of the biochemical reactions in which homocysteine is involved, deficiencies of vitamin B6, folic acid (vitamin B9), and vitamin B12 can lead to high homocysteine levels (Miller et al., The American journal of clinical nutrition 59 (5): 1033-9 (1994)). Hyperhomocysteinemia is typically managed with vitamin B6, folic acid, and vitamin B12 supplementation (van Guldener and Stehouwer, Expert Opinion on Pharmacotherapy 2 (9): 1449-1460 (2001)). Taurine supplementation also has been found to reduce homocysteine levels (Ahn, Adv. Exp. Med. Biol. 643:415-22 (2009)).

An inadequate intake of B vitamins, as well as genetic factors that affect the body's absorption and use of folic acid, can lead to elevated homocysteine levels. Other contributors to elevated homocysteine levels include stress and coffee consumption. The stress-induced neurotransmitters epinephrine and norepinephrine are metabolized in the liver via a process that uses methyl groups. This can also increase the need for folic acid. In addition, elevated homocysteine levels may be due to low levels of thyroid hormone, kidney disease, psoriasis, and side effects of some medications.

Treatment of hyperhomocysteinemia can include treatments to reduce the levels of homocysteine, treatments for diseases associated with high levels of homocysteine, treatment to reduce the risk of developing a disease associated with high levels of homocysteine, or a combination thereof. The treatment for elevated homocysteine is to take steps to lower the levels by increasing intake of B vitamins by, for example, eating more green leafy vegetables, fruits, and grain-based foods fortified with folic acid, and by reducing stress in the subject. The richest food sources of folate (the form of folic acid found in food) are green vegetables, orange juice, and beans. Folic acid intake can also be increased by taking a multivitamin or other pill that provides 400 micrograms of folic acid. Stress can be reduced by, for example, practicing breathing exercises, meditation, and mind-body exercises such as yoga. Reducing foods high in animal protein can also help lower homocysteine levels. Where another source of high homocysteine levels is indicated, such as low levels of thyroid hormone, kidney disease, psoriasis, and a side effect of a medication, treatment can include treatments directed to such indications.

Fluorescence can be measured with a variety of instrumentation compatible with a wide range of assay formats. For example, spectrofluorimeters have been designed to analyze microtiter plates, microscope slides, printed arrays, cuvettes, etc. See Principles of Fluorescence Spectroscopy, by J. R. Lakowicz, Springer Science+Business Media, Inc., 3^{rd} Ed., 2010; Chemiluminescence and Bioluminescence: Past, Present and Future, by A. Roda, editor, Royal Society of Chemistry, 2010; and Bioluminescence & Chemiluminescence: Progress & Current Applications; Philip E. Stanley and Larry J. Kricka, editors, World Scientific Publishing Company, January 2002.

The disclosed methods include the determination, identification, indication, correlation, diagnosis, prognosis, etc. (which can be referred to collectively as “identifications”) of subjects, diseases, conditions, states, etc. based on measurements, detections, comparisons, analyses, assays, screenings, etc. For example, subjects as at risk of disease can be identified based on detection of high levels of homocysteine in the subject. Such identifications are useful for many reasons. For example, and in particular, such identifications allow specific actions to be taken based on, and relevant to, the particular identification made. For example, diagnosis of a particular disease or condition in particular subjects (and the lack of diagnosis of that disease or condition in other subjects) has the very useful effect of identifying subjects that would benefit from treatment, actions, behaviors, etc. based on the diagnosis. For example, treatment for a particular disease or condition in subjects identified is significantly different from treatment of all subjects without making such an identification (or without regard to the identification). Subjects needing or that could benefit from the treatment will receive it and subjects that do not need or would not benefit from the treatment will not receive it.

Accordingly, also disclosed herein are methods comprising taking particular actions following and based on the disclosed identifications. For example, disclosed are methods comprising creating a record of an identification (in physical—such as paper, electronic, or other—form, for example). Thus, for example, creating a record of an identification based on the disclosed methods differs physically and tangibly from merely performing a measurement, detection, comparison, analysis, assay, screen, etc. Such a record is particularly substantial and significant in that it allows the identification to be fixed in a tangible form that can be, for example, communicated to others (such as those who could treat, monitor, follow-up, advise, etc. the subject based on the identification); retained for later use or review; used as data to assess sets of subjects, treatment efficacy, accuracy of identifications based on different measurements, detections, comparisons, analyses, assays, screenings, etc., and the like. For example, such uses of records of identifications can be made, for example, by the same individual or entity as, by a different individual or entity than, or a combination of the same individual or entity as and a different individual or entity than, the individual or entity that made the record of the identification. The disclosed methods of creating a record can be combined with any one or more other methods disclosed herein, and in particular, with any one or more steps of the disclosed methods of identification.

As another example, disclosed are methods comprising making one or more further identifications based on one or more other identifications. For example, particular treatments, monitorings, follow-ups, advice, etc. can be identified based on the other identification. For example, identification of a subject as having a disease or condition with a high level of a particular component or characteristic can be further identified as a subject that could or should be treated with a therapy based on or directed to the high level component or characteristic. A record of such further identifications can be created (as described above, for example) and can be used in any suitable way. Such further identifications can be based, for example, directly on the other identifications, a record of such other identifications, or a combination. Such further identifications can be made, for example, by the same individual or entity as, by a different individual or entity than, or a combination of the same individual or entity as and a different individual or entity than, the individual or entity that made the other identifications. The disclosed methods of making a further identification can be combined with any one or more other methods disclosed herein, and in particular, with any one or more steps of the disclosed methods of identification.

As another example, disclosed are methods comprising treating, monitoring, following-up with, advising, etc., a subject identified in any of the disclosed methods. Also disclosed are methods comprising treating, monitoring, following-up with, advising, etc., a subject for which a record of an identification from any of the disclosed methods has been made. For example, particular treatments, monitorings, follow-ups, advice, etc., can be used based on an identification and/or based on a record of an identification. For example, a subject identified as having a disease or condition with a high level of a particular component or characteristic (and/or a subject for which a record has been made of such an identification) can be treated with a therapy based on or directed to the high level component or characteristic. Such treatments, monitorings, follow-ups, advice, etc., can be based, for example, directly on identifications, a record of such identifications, or a combination. Such treatments, monitorings, follow-ups, advice, etc. can be performed, for example, by the same individual or entity as, by a different individual or entity than, or a combination of the same individual or entity as and a different individual or entity than, the individual or entity that made the identifications and/or record of the identifications. The disclosed methods of treating, monitoring, following-up with, advising, etc. can be combined with any one or more other methods disclosed herein, and in particular, with any one or more steps of the disclosed methods of identification.

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

The term “monitoring” as used herein refers to any method in the art by which an activity or process, such as the course of a disease or the course of treatment of a disease, can be measured.

The term “providing” as used herein refers to any means of adding a compound or molecule to something known in the art. Examples of providing can include the use of pipettes, pipettemen, syringes, needles, tubing, guns, etc. This can be manual or automated. It can include transfection by any mean or any other means of providing nucleic acids to dishes, cells, tissue, cell-free systems and can be in vitro or in vivo.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the compounds of the invention.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

A cell can be in vitro. Alternatively, a cell can be in vivo and can be found in a subject. A “cell” can be a cell from any organism including, but not limited to, a bacterium.

### Examples

### Example 1: Functional Characteristics of Fluorescent Probe Selective for Homocysteine

During a search for redox-based fluorescent probes for H₂S (Peng et al., Angew Chem Int Edit, 2011, 50, 9672-9675), a fluorescent probe (DN-2) was discovered with selectivity towards Hey over other biological thiols such as cysteine and glutathione in aqueous media. Thiols show reducing ability and help to maintain the redox states in biological systems. Redox reactions have been employed in thiol detections. For example, Ellman's reagent (5,5′-dithiobis-2-nitrobenzoic acid or DTNB; Ellman, Arch. Biochem. Biophys., 1959, 82, 70-77) is widely used in the quantitation of sulfhydryl groups through the reductive cleavage of a disulfide bond, which releases the chromogenic indicator. However, only a few studies have been reported on the difference in reducing ability among thiols (Hogg, Free Radical Bio Med, 1999, 27, 28-33; Gabaldon, Arch. Biochem. Biophys., 2004, 431, 178-188).

Dansyl derivative 1 (DN-2) was synthesized as a fluorescent probe. The synthesis of DN-2 is accomplished in 3 steps from commercially available starting materials (Scheme 1). The substitution reaction between 5-naphthol-1-sulfonate 3 and ethylene diamine was followed by reductive amination, which installed three methyl groups on the amino groups (O'Connor et al., Org. Lett., 2006, 8, 1581-1584). Then the sulfonate was converted to sulfonyl chloride, which reacted quantitatively with sodium azide to form the sulfonyl azide, DN-2 (1). This short and convenient synthetic route has provided the probe in a 37% isolated yield.

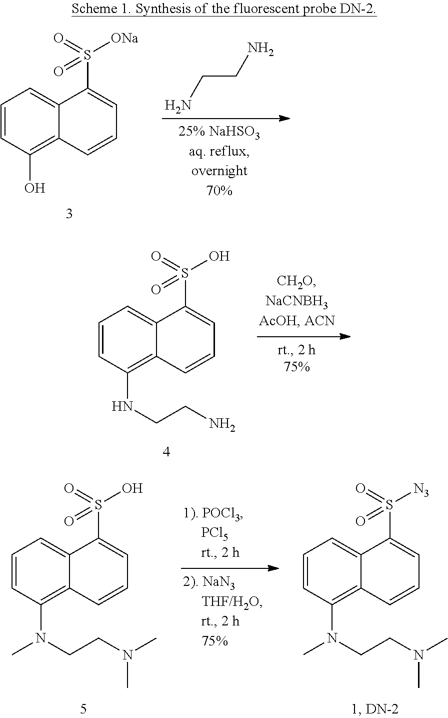

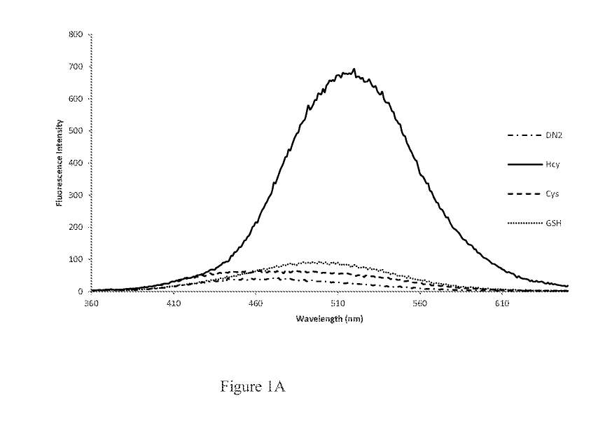

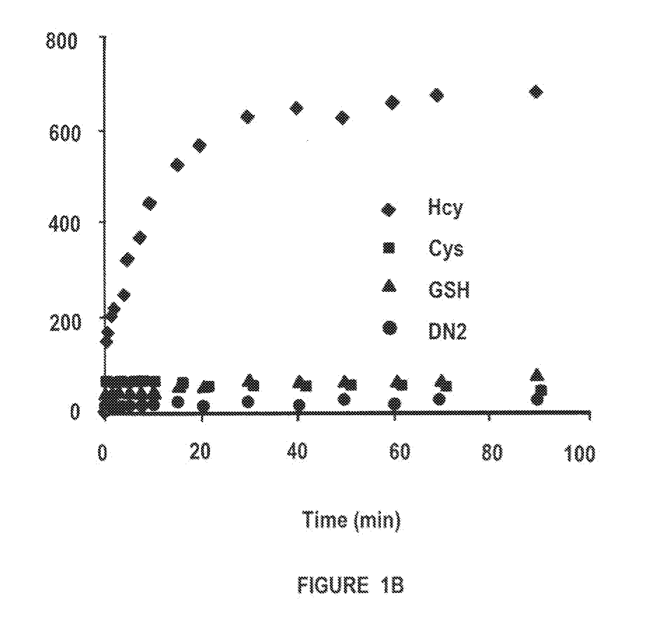

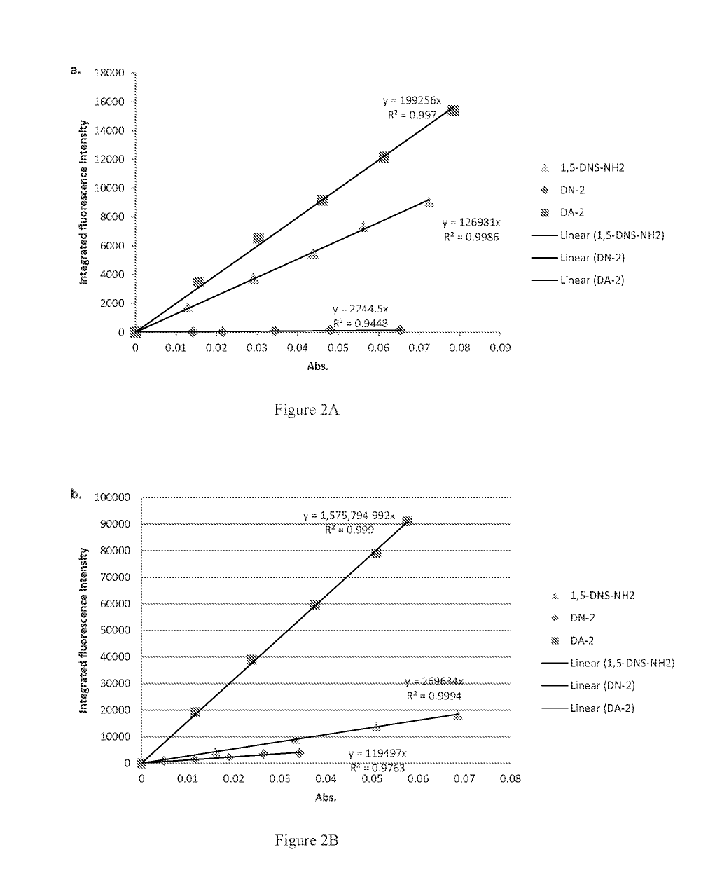

Fluorescence spectroscopy was used to monitor the reaction between DN-2 and biological thiols, Cys, Hey, and GSH. As is shown in FIG. 1A, a significant fluorescence increase (˜25-fold) at around 517 nm was observed for homocysteine. Such fluorescent changes are also readily visible to the naked eyes. However, with Cys and GSH, only 2 and 3-fold fluorescence intensity change was observed, respectively. Spectroscopic (¹H NMR, ¹³C NMR, and MS) identification of the product confirmed that the sulfonyl azide was reduced by homocysteine, forming the corresponding sulfonamide and homocystine (Scheme 2). The fluorescence increase was due to the formation of highly fluorescent DA-2 ((Φ_{FL} (DN-2)=0.007 and Φ_{FL} (DA-2)=0.58 in acetonitrile, Φ_{FL} (DN-2)=0.02 and Φ_{FL} (DA-2)=0.29 in water, FIG. 2).

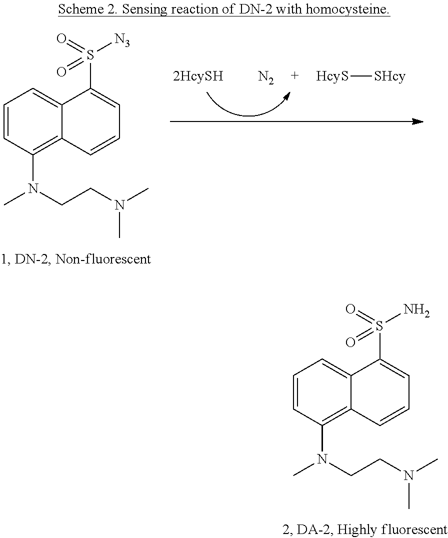

The reaction time profile of DN-2 was compared to the reaction time profile of thiols. Specifically, DN-2 was dissolved in the solution to a final concentration of 120 μM, while thiol concentrations were 100 μM. The probe, DN-2, was only weakly fluorescent in solvent systems, including acetonitrile (ACN), 100 mM sodium phosphate buffer at pH 7.4, and water. Immediately after the addition of thiols, an initial small increase in fluorescence was observed for all species (FIG. 1B). However, the fluorescent intensity quickly reached a plateau for Cys and GSH. For Hcy, the fluorescence intensity reached a plateau at about the 1 hour point at a much higher level. The selectivity for Hcy over Cys and GSH was found to be 8-12-fold. However, since GSH concentration in plasma is as low as ˜2 μM (Jones et al., Clin. Chim. Acta, 1998), one would not expect interference problem with GSH.

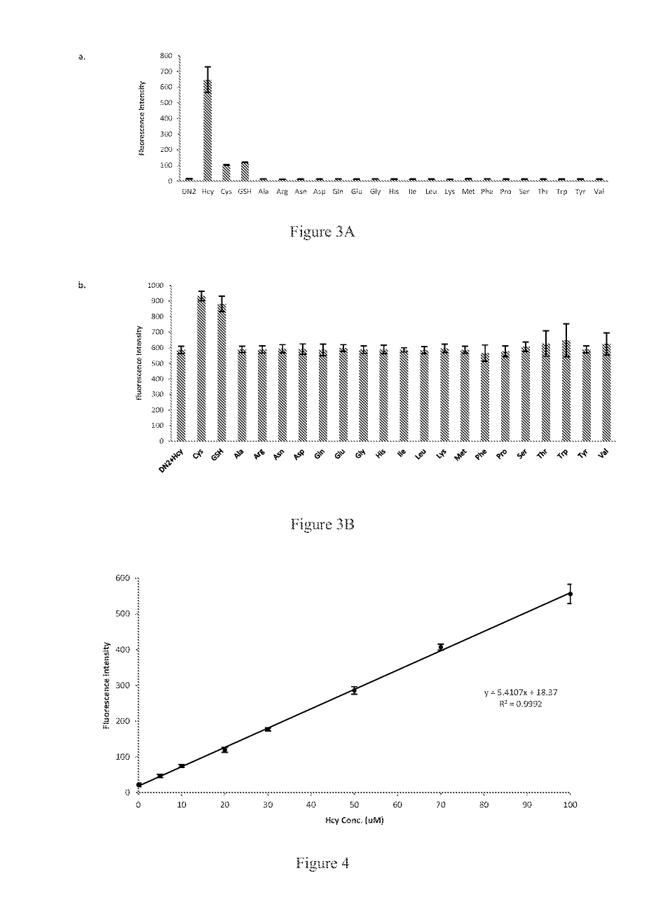

To further test the feasibility of DN-2 as a fluorescent probe in a complex biological system, the selectivity of DN-2 was tested among various amino acids in phosphate buffer with 10% ethanol (FIG. 3A). It was found that DN-2 did not respond to most amino acids. A selectivity of over 45-fold was found among these amino acids. The reaction between DN-2 and homocysteine was carried out in the presence of various amino acids (100 μM) (FIG. 3B) in order to test how the presence of other amino acids affects the fluorescence response of DN-2 to homocysteine. It was found that the fluorescence response of DN-2 to homocysteine was elevated by ˜40% in the presence of 100 μM cysteine. GSH showed similar effect. In contrast, none of other amino acids affected the detection of homocysteine.

A fluorescent probe is especially useful for quantitation if a linear calibration curve could be obtained. Therefore, experiments were performed to obtain a calibration curve using a series of concentrations for homocysteine. As expected, a linear calibration curve with R²>0.99 was obtained for homocysteine (FIG. 4). This linearity is very important for quantitative analysis of homocysteine.

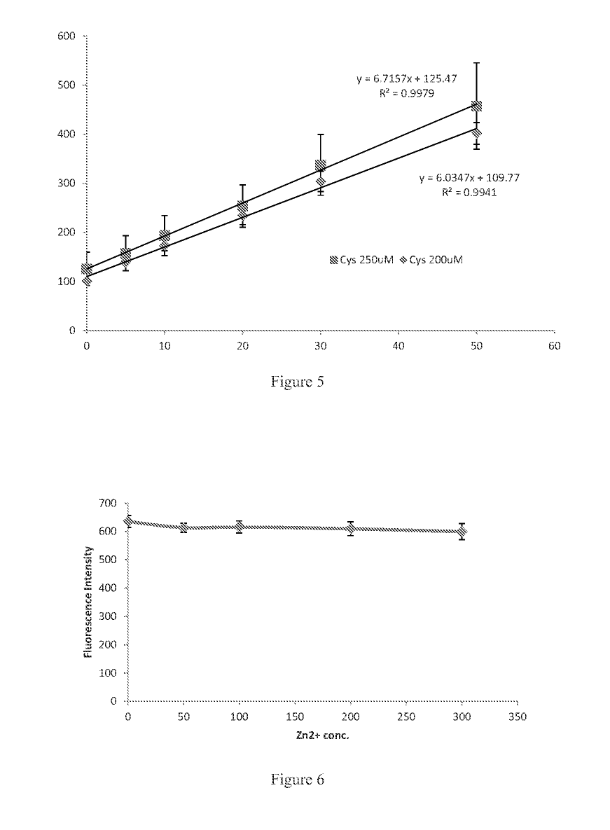

Since the presence of cysteine was found to increase the fluorescence response of DN-2 to homocysteine by ˜40%, it was tested whether endogenous cysteine fluctuations (˜200-250 μM) would affect homocysteine detection. In order to test the effect of cysteine fluctuation, calibration curves of homocysteine at 0-50 μM was generated in the presence of 200 and 250 μM of cysteine (FIG. 5). It was found that these two curves are very similar, though not identical. Therefore, the accuracy of homocysteine quantitation may be slightly affected by cysteine fluctuation. However, the selectivity of DN-2 to homocysteine still allows a good estimation of homocysteine level with large (˜50 μM) fluctuation of cysteine concentrations.

As arylsulfonyl azide was found to be a very sensitive fluorescent probe for sulfide (Chen et al., Chem Soc Rev, 2010, 39, 2120-2135; Wang et al., J. Fluoresc., 2013, DOI: 10.1007/s10895-013-1296-5). DN-2 also reacts with sulfide. In order to exclude the influence of sulfide in the selective detection of Hcy, a solution of ZnCl₂ could be added into the reaction buffer to precipitate sulfide out of the solution in the form of ZnS (Kₛₚ(ZnS)-2×10⁻²⁵). It was found that the presence of high concentration of Zn²⁺ (at 300 μM) shows very little interference with the fluorescence response of DN-2 to Hcy (FIG. 6).

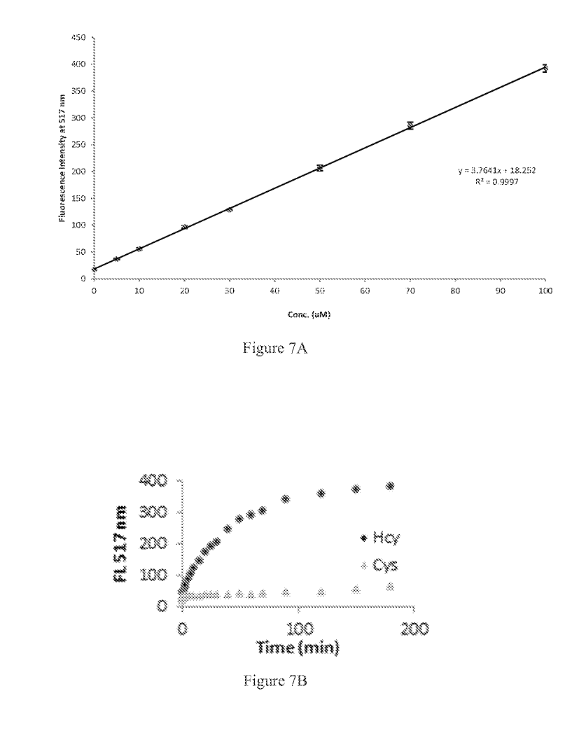

Due to the significance of Hcy in biological systems, the application of this probe in serum was examined. In diluted (10%) deproteinized fetal bovine serum (FBS), with addition of Hcy, the DN-2 probe selectively responded to Hcy over Cys (FIG. 7B). Furthermore, concentration-dependent fluorescence changes of DN-2 in diluted deproteinized FBS were observed. A linear calibration curve (R²=0.9997) was obtained showing that DN-2 can be used to detect and quantitate Hcy in serum. The detection limit of Hcy in deproteinized FBS was 10 μM based on a 3-fold signal-to-noise ratio.

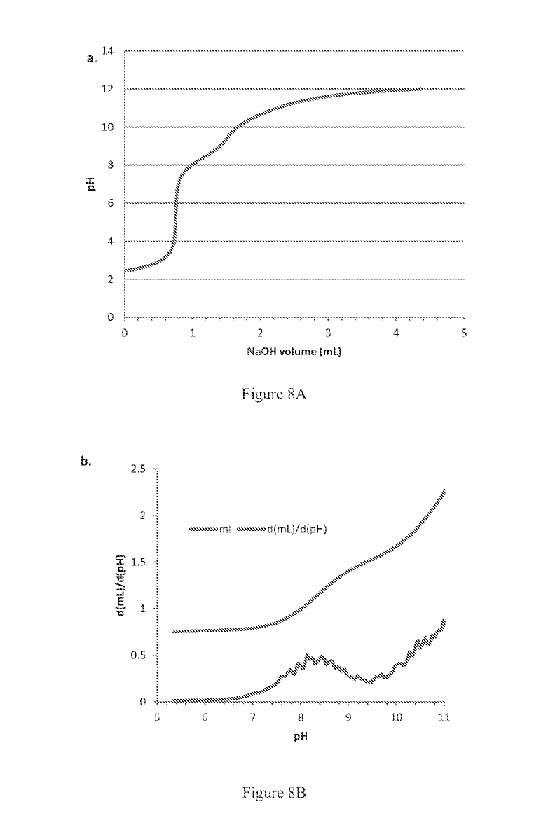

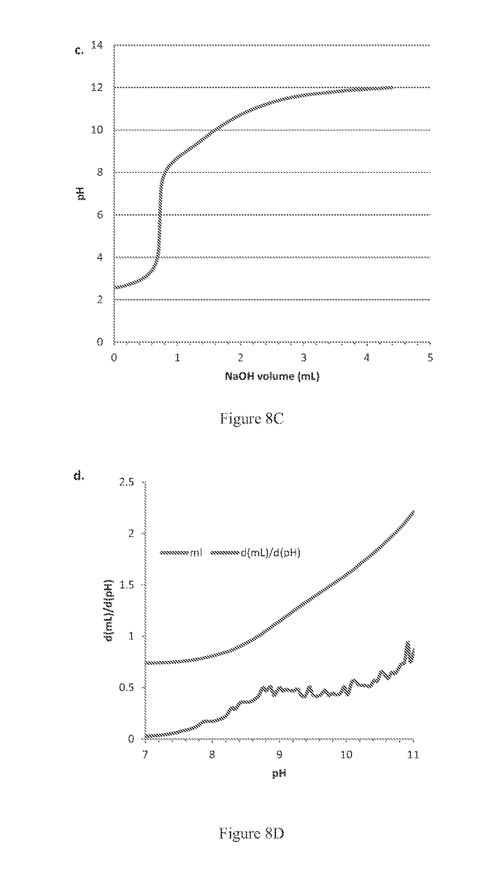

The experiments showed that DN-2 selectively oxidizes homocysteine in buffer and diluted deproteinized FBS. Although cysteine can also be reduced by DN-2 at very high concentrations (100 mM), there is apparently a large difference at lower concentrations. Thus, experiments were conducted to help understand the difference in reducing ability between cysteine and homocysteine. Homocysteine has one extra methylene group compared to cysteine. However, this does not make its sulfhydryl group a stronger nucleophile since most nucleophilic substitution-based fluorescent probes show similar reactivity with both thiols. The pKa of the sulfhydryl groups on cysteine and homocysteine were examined using pH titration (Friedman et al., J Am Chem Soc, 1965, 87, 3672-3682). It was found that the pKa was 8.25 for cysteine and 8.9 for homocysteine (FIG. 8). The results are similar to previously reported values (Friedman et al., J Am Chem Soc, 1965, 87, 3672-3682). According to their pKa, cysteine should be deprotonated more easily than homocysteine. Therefore, one would expect that cysteine being a stronger reducing agent than homocysteine if pKa was the determining factor. However, this is not the case in the redox reaction between these thiols with the probe DN-2. In fact, it has been reported that oxidation of homocysteine by albumin is faster than that of cysteine and the oxidation is accelerated by basic conditions or addition of a copper catalyst (Gabaldon, Arch. Biochem. Biophys., 2004, 431, 178-188).

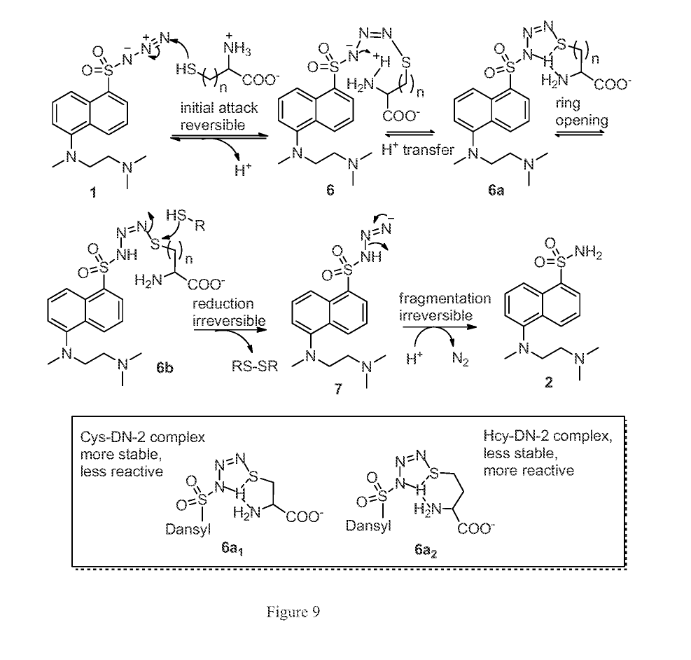

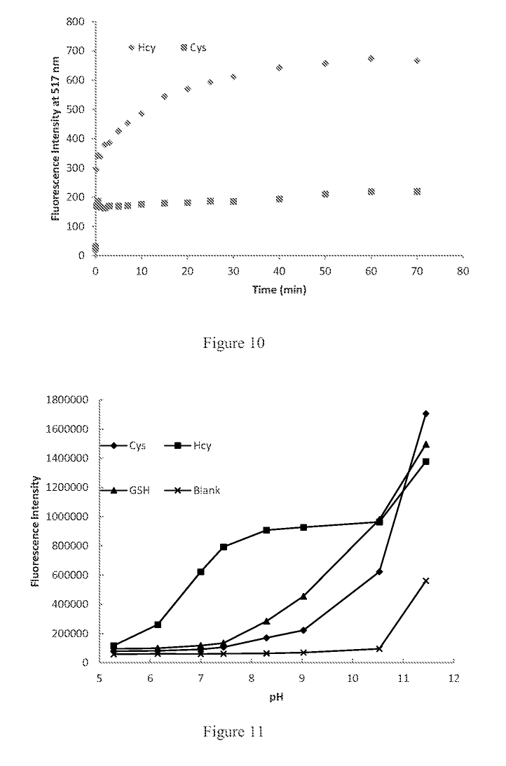

The reduction mechanism of an azido compound by a dithiol has been proposed in a previous report (Cartwright et al., Nucleic Acids Res, 1976, 3, 2331-2340). Based on the experimental results, it is considered that the initial attack by thiol leads to the formation of an intermediate 6 (FIG. 9), which is responsible for the initial 2-3-fold fluorescence increase. Proton transfer then occurs and facilitates the formation of a hydrogen bond-based bicyclic intermediate 6a (FIG. 9), which is in equilibrium with the ring opening form 6b. Reduction of 6b by another molecule of thiol releases molecular nitrogen to give the final product 2. The stability of the bicyclic transition state 6a is considered responsible for the difference in reactivity between the intermediates formed with Cys and Hcy, respectively. For this, the fused five-membered ring formed in the Cys complex (6a₁) leads to an enhanced stability compared to the six-membered ring formed in the Hcy complex (6a₂). Thus the reduction of the Hcy complex is easier, leading to the formation of the final sulfonamide product (2). In addition, the reversibility of the initial step means that the intermediate (6) exists in equilibrium with the starting materials and thus the probe (1) is available to react with Hcy even in the presence of Cys. In fact, cyclic transition states/intermediates have been used to explain the mechanisms of other Cys- or Hcy-selective probes (Yang et al., Angew. Chem. Int. Ed., 2011, 50, 10690-10693; Strongin et al., J Am Chem Soc, 2004, 126, 3400-3401; Rusin et al., J Am Chem Soc, 2005, 127, 15949-15958). It is entirely possible that there are other conformational factors, which affect the reactivity of intermediate 6. In the fourth step, another thiol comes to attack the sulfur to form a disulfide. Although gaseous diatomic bond energy for general S—N bond is higher than S—S bond (Kerr, in CRC Handbook of Chemistry and Physics 1999-2000: A Ready-Reference Book of Chemical and Physical Data (CRC Handbook of Chemistry and Physics, D. R. Lide, (ed.), CRC Press, Boca Raton, Fla., USA, 2000), the formation of disulfide bond from the attack of sulfhydryl group on an electron-deficient S—N bond has been reported previously (Bao and Shimizu, Tetrahedron, 2003, 59, 9655-9659). In this step, it is conceivable that when excessive amount of cysteine exists in the reaction media, a Cys-Hcy mixed disulfide could also form. This explains why Cys alone does not trigger fluorescence increase but causes a ˜40% increase in fluorescence response to Hcy. Another dansyl azide analogue, 1,5-DNS-Az, was also shown to be selective for homocysteine (FIG. 10).

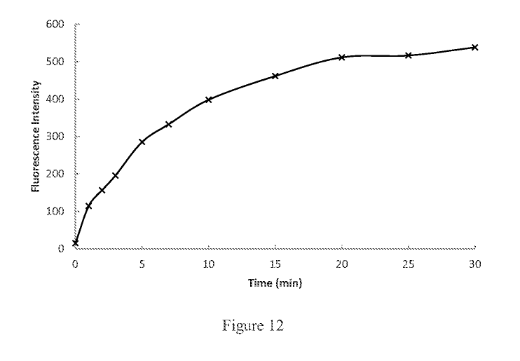

In addition, the influence of pH on the selectivity was tested using 96-well plate and a microplate reader (FIG. 11). Sodium phosphate buffer (100 mM) in the pH range of 5.3-11.5 was used in the experiments. A pH dependent fluorescence response was observed for DN-2 in the presence of thiols. It was found that DN-2 itself remains fluorescently stable at pH values lower than 10.5. The selectivity for homocysteine increased with increasing pH from 5-7.4. In buffers at pH higher than 7.4, reactions with cysteine and GSH were promoted. Therefore, the highest selectivity was observed at pH 7.4, which is the physiological pH.

The reaction time profile in phosphate buffer at 37° C. was also examined (FIG. 12). The result indicates that heating facilitated the reaction. The reaction time for 120 μM of DN-2 and 100 μM of homocysteine decreased from 60 min at room temperature (about 22° C.) to 30 min at 37° C.

### Experimental Details

General Information

Solvents and reagents were purchased from VWR International, Oakwood Product Inc., or Sigma-Aldrich Co. and used without purification unless specified otherwise. When necessary, solid reagents were dried under high vacuum. Reactions with compounds sensitive to air or moisture were performed under argon. Solvent mixtures are indicated as volume/volume ratios. Thin layer chromatography (TLC) was run on Sorbtech W/UV254 plates (0.25 mm thick), and visualized under UV-light or by a Ce—Mo staining solution (phosphomolybdate, 25 g; Ce(SO₄)₂.4H₂O, 10 g; conc. H₂SO₄, 60 mL; H₂O, 940 mL) with heating. Flash chromatography was performed using Fluka silica gel 60 (mesh size: 0.040-0.063 mm) using a weight ratio of ca. 30:1 for silica gel over crude compound. ¹H and ¹³C-NMR spectra were recorded on a Bruker 400 spectrometer (400 and 100 μMHz, respectively) in deuterated chloroform (CDCl₃), methanol-d₄ (CΦ₃OD), and DMSO-d₆ with either tetramethylsilane (TMS) (0.00 ppm) or the NMR solvent as the internal reference. UV-Vis absorption spectra were recorded on a Shimadzu PharmaSpec UV-1700 UV-Visible spectrophotometer. Fluorescence spectra were recorded on a Shimadzu RF-5310PC spectrofluorophotometer. 96-Well plates were read and recorded on a PerkinElmer 1420 multi-label counter.

### Synthesis and Characterization

5-(2-Aminoethylamino)naphthalene-1-sulfonic acid 4 and 5-(N-(2-(dimethylamino)ethyl)-N-methylamino)naphthalene-1-sulfonic acid 5: was performed following literature reported procedures (O'Connor et al., Org Lett, 2006, 8, 1581-1584).

### 5-(N-(2-(Dimethylamino)ethyl)-N-methylamino)naphthalene-1-sulfonyl azide 1

To a Ar protected solution of 5 (110 mg, 0.36 mmol) in POCl₃ (570 μL, 6.2 mmol) was added PCl5 in 2 portions (430 mg, 2.1 mmol). The reaction was stirred on ice bath for 1 h, then warmed to room temperature, and stirred for additional 2 h. The reaction mixture was poured into 10 g ice. EtOAc extraction and evaporation gave crude intermediate 5-(N-(2-(dimethylamino)ethyl)-N-methylamino)naphthalene-1-sulfonyl chloride, which was dissolved in MeOH (2 mL) and added into a stirred solution of NaN₃ (243 mg, 3.75 mmol) in a 1:1 mixed solvent of MeOH/H₂O (4 mL). The reaction mixture was stirred at room temperature for 2 h. MeOH was evaporated and the product was extracted with EtOAc. The organic phase was washed with water and brine, and dried over Na₂SO₄. Solvent evaporation followed by flash chromatography (CH₂Cl₂:Hex:EtOAc, 1:2:0.2) gave a light yellow solid (90 mg, 75% yield). ¹H NMR (CDCl₃): 8.64 (d, J=8.4 Hz, 1H), 8.34 (d, J=7.2 Hz, 1H), 8.64 (d, J=8.8 Hz, 1H), 7.74-7.64 (m, 2H), 7.39 (d, J=7.6 Hz, 1H), 3.59 (t, J=7.0 Hz, 2H), 3.12 (t, J=7.0 Hz, 2H), 2.91 (s, 3H), 2.91 (s, 6H); ¹³C NMR (CDCl₃): 149.8, 134.1, 131.7, 131.0, 130.4, 129.7, 129.2, 124.0, 120.7, 118.3, 60.6, 51.1, 50.4, 45.1; IR 2916.9, 2421.7, 2130.7, 1571.2, 1465.2, 1166.6, 792.6, 743.8; MS (ES+) 333.9 (M+1)⁺.

### 5-(N-(2-(Dimethylamino)ethyl)-N-methylamino)naphthalene-1-sulfonyl amide 2

¹H NMR (CDCl₃): 8.51-8.46 (m, 2H), 8.26 (d, J=7.2 Hz, 1H), 7.65-7.57 (m, 2H), 7.40-7.38 (m, 1H), 3.52 (t, J=6.0 Hz, 2H), 3.13 (t, J=6.0 Hz, 2H), 2.84 (s, 3H), 2.66 (s, 6H); ¹³C NMR (CDCl₃): 149.8, 139.1, 130.6, 129.5, 128.7, 127.5, 127.0, 123.7, 120.8, 117.3, 60.1, 50.2, 49.9, 43.5; MS (ES+) 308.0 (M+1)⁺.

Relative Quantum Yield Determination of DN-2 (1) and DA-2 (2)

1,5-DNS-NH₂ was used as the reference for relative quantum yield determination. Absorption and emission (λ_{Ex}=325 nm) spectra were recorded for a series of concentrations (16 μM, 12 μM, 8 μM, 4 μM and 0 μM in acetonitrile or deionized water) of 1,5-DNS-NH₂, DN-2 (1) and DA-2 (2). Integrated fluorescence intensity was plotted against the absorption values at each concentration (FIGS. 11 and 12) (Williams et al., Analyst, 1983, 108, 1067-1071). Relative quantum yield values can be calculated using slopes of each compound.

Determination of the pKa Values of the Sulhydryl Groups on Cysteine and Homocysteine

Automatic pH titration on a (Accumet® Research, AR10 pH meter) was used to determine the pKa values of the sulfhydryl groups on cysteine and homocysteine. NaOH solution (99.35 mM) was standardized with potassium hydrogen phthalate (KHP) and used for titration of 5 mM cysteine hydrochloride and 5 mM homocysteine hydrochloride. Titration curve was created and analyzed using TitriSoft 2.51. Experiment was triplicated to obtain the average pKa values (8.25 for cysteine and 8.9 for homocysteine).

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a fluorescent probe” includes a plurality of such fluorescent probes, reference to “the fluorescent probe” is a reference to one or more fluorescent probes and equivalents thereof known to those skilled in the art, and so forth.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. may include numerous options and alternatives, this should not be construed as, and is not an admission that, such options and alternatives are equivalent to each other or, in particular, are obvious alternatives. Thus, for example, a list of different compounds does not indicate that the listed compounds are obvious one to the other, nor is it an admission of equivalence or obviousness.

Those skilled in the art will recognize, or be able to ascertain using no more than routine experimentation, many equivalents to the specific embodiments of the method and compositions described herein. Such equivalents are intended to be encompassed by the following claims.

1. A method of measuring the amount of homocysteine in a sample, the method comprising:
incubating a sample and a sulfonyl azide compound of formula II 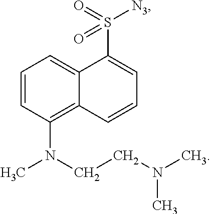 [SMILES: CN(C)CCN(C)c1cccc2c(S(=O)(=O)N=[N+]=[N-])cccc12]
and
measuring fluorescence of a reaction product of the compound and homocysteine, wherein the measured fluorescence level indicates the amount of homocysteine in the sample.

2. The method of claim 1, wherein the sample is a body fluid sample of a subject.

3. The method of claim 2, wherein the body sample fluid is serum.

4. The method of claim 3, wherein the serum is exposed to reducing conditions prior to the incubation.

5. The method of claim 4, wherein the exposure to reducing conditions is accomplished by exposing the serum to gel containing tris(2-carboxyethyl)phosphine) (TCEP).

6. The method of claim 3, wherein the serum is diluted prior to the incubation.

7. The method of claim 1, wherein the incubation is in the presence of a transition metal salt.

8. The method of claim 1, wherein the incubation is in the presence of a Zinc salt, a Cadmium salt, a Copper salt, a Lead salt, a Mercury salt, a Silver salt, or a combination thereof.

9. The method of claim 8, wherein the incubation is in the presence of a Zinc(II) salt, a Cadmium(II) salt, a Copper(II) salt, a Lead(II) salt, a Mercury(II) salt, a Silver(I) salt, or a combination thereof.

10. The method of claim 8, wherein the Zinc salt is ZnCl₂.

11. The method of claim 1, wherein the incubation and measurement is carried out at a pH of 6 to 9.

12. The method of claim 11, wherein the incubation and measurement is carried out at a pH of 7 to 8.

13. The method of claim 12, wherein the incubation and measurement is carried out at a pH of 7.4.

14. The method of claim 1, wherein the measurement is carried out at a pH of 7.4.

15. The method of claim 1, wherein the measured fluorescence level indicates the amount of homocysteine in the sample by comparing the measured fluorescence level to calibration curve of the fluorescence level of different concentrations of homocysteine.

16. The method of claim 1 further comprising identifying the subject as at risk of disease based on the indicated amount of homocysteine in the sample.

17. The method of claim 16, wherein the disease is cardiovascular disease or Alzheimer's disease.

18. The method of claim 16 further comprising treating the subject for the disease or to reduce the risk of the disease if the subject is identified as at risk of disease based on the indicated amount of homocysteine in the sample.

19. The method of claim 16, wherein the subject is identified as at risk of disease if the indicated amount of homocysteine in the sample is greater than 14 μmoles/liter.

20. The method of claim 19, wherein the subject is identified as at intermediate risk of disease if the indicated amount of homocysteine in the sample is greater than 29 μmoles/liter.

21. A kit for measuring the amount of homocysteine in a sample, the kit comprising:
a sulfonyl azide compound of formula II 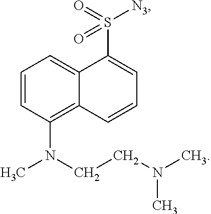 [SMILES: CN(C)CCN(C)c1cccc2c(S(=O)(=O)N=[N+]=[N-])cccc12]
and
a transition metal salt.

22. The kit of claim 21, wherein the incubation is in the presence of a Zinc salt, a Cadmium salt, a Copper salt, a Lead salt, a Mercury salt, a Silver salt, or a combination thereof.

23. The kit of claim 22, wherein the incubation is in the presence of a Zinc(II) salt, a Cadmium(II) salt, a Copper(II) salt, a Lead(II) salt, a Mercury(II) salt, a Silver(I) salt, or a combination thereof.

24. The kit of claim 22, wherein the Zinc salt is ZnCl₂.

25. A compound of formula II: 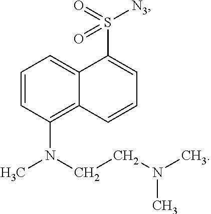 [SMILES: CN(C)CCN(C)c1cccc2c(S(=O)(=O)N=[N+]=[N-])cccc12]
